(19) United States
(12) Patent Application Publication
(10) Pub. No.: US 2024/0122812 A1
(43) Pub. Date: Apr. 18, 2024

(54) GASTROSTOMY TUBE DEVICES AND METHODS FOR USE

(71) Applicant: THE BOARD OF TRUSTEES OF THE LELAND STANFORD JUNIOR UNIVERSITY, Stanford, CA (US)

(72) Inventors: David Goldenberg, Palo Alto, CA (US); Ivan Tzvetanov, Kailua-Kona, HI (US)

(21) Appl. No.: 18/389,679

(22) Filed: Dec. 19, 2023

Related U.S. Application Data
(63) Continuation of application No. PCT/US22/34337, filed on Jun. 21, 2022.
(60) Provisional application No. 63/213,060, filed on Jun. 21, 2021.

(51) Int. Cl. A61J 15/00

(52) U.S. Cl. CPC A61J 15/0015; A61J 15/0034; A61J 15/0049; A61M 25/10185

(57) ABSTRACT

Gastrostomy tubes and systems and methods for implanting them are provided. In one example, the gastrostomy tube includes an elongate tubular member including a first end sized for introduction into the patient's mouth, a second end including an expandable member or other bumper, and a feeding lumen extending between the first and second ends. A bolster is connectable around the first end after the first end has been directed from within the patient's stomach through intervening tissue and extends from the patient's skin and the bumper is placed against the wall of the stomach. An adapter is connectable to the bolster after the first end and excess material of the tubular member beyond the bolster has been separated, the adapter including a connector for removably connecting to a feeding tube.

### RELATED APPLICATION DATA

The present application is a continuation of International Application No. PCT/US/2022/034337, filed Jun. 21, 2022, which claims benefit of U.S. provisional application Ser. No. 63/213,060, filed Jun. 21, 2021, the entire disclosures of which are expressly incorporated by reference herein.

### TECHNICAL FIELD

The present invention relates to medical devices and, more particularly to gastrostomy tube devices and to systems and methods for implanting and using such devices.

### BACKGROUND

Gastrostomy tubes are long tubes that extend from a patient's abdomen. They are placed when a patient cannot take in nutrients by mouth, e.g., for at least thirty days. Low profile tubes are available that are flush against the patient's abdomen; however, they cannot be safely placed until a tract is formed from the patient's stomach to their skin using a traditional long gastrostomy tube, such as that shown in FIG. 1. These long gastrostomy tubes can cause multiple problems including:
1) Feeding tubes can be pulled out accidentally, e.g., by delirious patients that are critically ill in the hospital. Accidental removal of this tube can be a catastrophic event requiring emergency surgery, and, therefore, patients are often placed in restraints that carry significant risk and cost;
2) Tubes are unsightly and psychologically triggering to patients;
3) Long tubes are more prone to leaking and can be cumbersome to patients; and
4) There is no way to convert a traditional long tube to a low profile feeding tube.

Therefore, there is a need for improved gastrostomy tubes that avoid problems associated with conventional gastrostomy tubes.

### SUMMARY

The present invention is directed to medical devices and, more particularly to gastrostomy tube devices and to systems and methods for implanting and using such devices.

A low-profile gastrostomy tube is described herein that can be placed during an initial procedure instead of waiting, e.g., three months, after a traditional long gastrostomy tube has been placed. In an exemplary method, the gastrostomy tube may be implanted using the following procedure:

Step 1. A gastrostomy tube with bumper is engaged. This may occur with a balloon being inflated or with a bistable mechanism activated. The balloon may include a one-way valve to ensure that the balloon remains remain inflated after the tube is cut to size. Alternatively, the bumper may be a fixed shape that is always expanded.

Step 2. Under endoscopic visualization, a guidewire is introduced into the subject's stomach through the skin, e.g., after appropriate insufflation of the stomach.

Step 3. The guidewire is pulled out the subject's mouth and attached to the gastrostomy tube. The tube is then pulled down the mouth and out the stomach with the guidewire.

Step 4. A bolster is placed around the tube to secure the exposed end of the tube to the abdomen. The tube is secured as the abdominal wall is sandwiched between the intragastric bumper and the extragastric bolster.

Step 5. The tube is cut such that it is flush with the bolster.

Step 6. An adapter is secured to the bolster. In one example, this may be done using a flair fitting connection with threaded connection between the bolster and adapter. This may also be done using other mechanisms for connecting the two pieces to achieve a mechanical fit, including, but not limited to a snap attachment, adhesive, screws, pins, magnets, and the like. The adapter may secure the bolster in order to allow for manipulation of the intragastric bumper. In case of a balloon, there is a portal to deflate or inactivate the balloon. In case of a bistable mechanism, there is a mechanism for changing the conformation of the bumper for possible removal of the tube. In case of a fixed shape, the intragastric bumper is not manipulated.

Step 7. A feeding attachment is attached to the adapter when the tube feed is in use and secured to the adapter. The attachment is secured mechanically in such a way that pulling the tube will dislodge from the adapter and the gastrostomy tube will not be pulled out of the abdomen. This may be accomplished using one or more of a secure fitting, rotation, and the like.

Step 8. After the tube is no longer needed, the intragastric bumper is deactivated. If a balloon is used, the balloon may be deflated by aspirating through the adapter/bolster unit or popped. If the bumper includes a bistable device, the device may be compressed, collapsed, or otherwise adjusted through the adapter/bolster unit so the bumper may be removed from the skin.

Alternatively, a long gastrostomy feeding tube may be converted to a low-profile tube using the devices and methods herein. In this alternative, an annular bolster may be placed around a long gastrostomy tube, e.g., against the patient's abdomen, which secures the tube firmly against the abdominal skin. The tube is then cut to remove any excess material beyond the bolster, and an adapter is secured to the bolster, e.g., as described above.

The gastrostomy tube devices herein can also be placed into existing gastrocutaneous tracts or fistulas, e.g., for tube replacements. After an old tube is removed, a patent tract may remain in the patient's abdomen from the skin to the stomach. In an exemplary method, a replacement gastrostomy tube including a bumper on a first end may be implanted using the following procedure:

Step 1. The bumper is manipulated, e.g., deflated if a balloon, or compressed if a bistable mechanism, to ensure that the intragastric bumper is flush against the outer surface of the tube.

Step 2. The first end of the tube (and bumper) is then inserted through the skin to the stomach via the gastrocutanenous fistula.

Step 3. The intragastric bumper is then expanded (e.g., either via balloon or bistable mechanism).

Step 4. A bolster is then placed around the tube to secure the exposed end of the tube to the abdomen. The tube is secured as the abdominal wall is sandwiched between the intragastric bumper and the extragastric bolster.

Step 5. The tube is cut such that the exposed end is flush with the bolster.

Step 6. An adapter is secured to the bolster. In one example, this may be done using a flair fitting connection with threaded connection between the bolster and adapter. This may also be done using other mechanisms for connecting the two pieces to achieve a mechanical fit, including, but not limited to a snap attachment, adhesive, screws, pins, magnets, and the like. The adapter may secure the bolster in order to allow for manipulation of the intragastric bumper. In case of a balloon, there is a portal to deflate or inactivate the balloon. In case of a bistable mechanism, there is a mechanism for changing the conformation of the bumper for possible removal of the tube.

In accordance with one example, a gastrostomy tube device is provided for implantation within an abdomen of a patient that includes an elongate tubular member comprising a first end sized for introduction into the patient's mouth, a second end including an expandable member or other bumper, and a feeding lumen extending between the first and second ends; a bolster connectable around the first end after the first end has been directed from within the patient's stomach through intervening tissue and extends from the patient's skin; and an adapter connectable to the bolster after the first end and excess material of the tubular member beyond the bolster has been separated, the adapter comprising a connector for removably connecting to a feeding tube.

In accordance with another example, a kit is provided for implanting a gastrostomy tube within an abdomen of a patient that includes an elongate tubular member comprising a first end sized for introduction into the patient's mouth, a second end including an expandable member or other bumper, and a feeding lumen extending between the first and second ends; a bolster connectable around the first end after the first end has been directed from within the patient's stomach through intervening tissue and extends from the patient's skin; and an adapter connectable to the bolster after the first end and excess material of the tubular member beyond the bolster has been separated, the adapter comprising a connector for removably connecting to a feeding tube.

In accordance with still another example, a system is provided for implanting a gastrostomy tube within an abdomen of a patient that includes an elongate tubular member comprising a first end sized for introduction into the patient's mouth, a second end including an expandable member, a feeding lumen extending between the first and second ends, and an inflation lumen extending from the first end and communicating with an interior of the expandable member; a bolster connectable around the first end after the first end has been directed from within the patient's stomach through intervening tissue and extends from the patient's skin; an adapter connectable to the bolster after the first end and excess material of the tubular member beyond the bolster has been separated, the adapter comprising a connector for removably connecting to a feeding tube; and a source of inflation media connectable to the inflation lumen for introducing inflation media into the interior to expand the expandable member.

In accordance with yet another example, a system is provided for implanting a gastrostomy tube within an abdomen of a patient that includes an elongate tubular member comprising a first end sized for introduction into the patient's mouth, a second end including a bumper formed from flexible, distensible material, and, a feeding lumen extending between the first and second ends; a bolster connectable around the first end after the first end has been directed from within the patient's stomach through intervening tissue and extends from the patient's skin; an adapter connectable to the bolster after the first end and excess material of the tubular member beyond the bolster has been separated, the adapter comprising a connector for removably connecting to a feeding tube; and an instrument sized for introduction into the feeding lumen to distend the bumper to reduce its outer cross section and facilitate removal of the tubular member.

In accordance with another example, a method is provided for implanting a gastrostomy tube into an abdomen of a patient that includes providing an elongate tubular member comprising a first end, a second end including an expandable member, and a lumen extending between the first and second ends; directing a first end of a guidewire through the abdomen of the patient from skin overlying the patient's stomach through intervening tissue into the stomach; directing the first end of the guidewire along the patient's upper gastrointestinal tract from the patient's stomach and out the patient's mouth; expanding the expandable member on the second end of the tubular member; introducing the first end of the tubular member using the guidewire into the patient's mouth and upper gastrointestinal tract until the tubular member is positioned within the patient's stomach; directing the first end of the tubular member through the intervening tissue until the first end is exposed outside the patient's abdomen; securing a bolster to the first end adjacent the patient's skin such that the intervening tissue is sandwiched between the expandable member within the stomach and the bolster; separating excess length of the tubular member exposed beyond the bolster; and securing an adapter to the bolster, the adapter comprising a connector for removably connecting a feeding tube to the adapter.

In accordance with still another example, a method is provided for implanting a gastrostomy tube into an abdomen of a patient that includes providing an elongate tubular member comprising a first end, a second end including a bumper, and a lumen extending between the first and second ends; directing a first end of a guidewire through the abdomen of the patient from skin overlying the patient's stomach through intervening tissue into the stomach; directing the first end of the guidewire along the patient's upper gastrointestinal tract from the patient's stomach and out the patient's mouth; introducing the first end of the tubular member using the guidewire into the patient's mouth and upper gastrointestinal tract until the tubular member is positioned within the patient's stomach; directing the first end of the tubular member through the intervening tissue until the first end is exposed outside the patient's abdomen and the bumper is positioned against the wall of the stomach; securing a bolster to the first end flush with the patient's skin such that the intervening tissue is sandwiched between the expandable member within the stomach and the bolster; separating excess length of the tubular member exposed beyond the bolster; and securing an adapter to the bolster, the adapter comprising a connector for removably connecting a feeding tube to the adapter.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 shows an example of a conventional gastrostomy tube implanted within a patient's abdomen.

FIG. 2A shows an example of a low profile gastrostomy tube device implanted within a patient's abdomen

FIGS. 2B and 2C are side and cross-sectional views, respectively, of the gastrostomy tube device of FIG. 2A.

FIGS. 3A and 3B show an example of a tubular body for a gastrostomy tube device, such as that shown in FIGS. 2A-2C, including a first end and an annular balloon on a second end with the balloon uninflated and inflated, respectively.

FIG. 3C shows the tubular body of FIGS. 3A and 3B with the balloon inflated and

showing internal lumens of the tubular body.

FIG. 3D is a cross-section of the tubular body of FIGS. 3A and 3B showing a central feeding lumen and an inflation lumen.

FIGS. 4A and 4B are side and top views, respectively, of an example of a bolster for a gastrostomy tube device, such as that shown in FIGS. 2A-2C, including connectors for securing the bolster to an end of the tubular body shown in FIGS. 3A and 3B.

FIGS. 5A and 5B are side and top views, respectively, of an example of an adapter for a gastrostomy tube device, such as that shown in FIGS. 2A-2C, that may be connected to the bolster of FIGS. 4A and 4B.

FIGS. 6A-6C show an exemplary method for implanting the gastrostomy tube device, such as that shown in FIGS. 2A-2C, within a subject's abdomen.

FIGS. 7A and 7B are cross-sectional views showing an example of a tubular body for a gastrostomy tube device, including a first end and a mechanically expandable bumper on a second end with the bumper collapsed and expanded, respectively.

FIG. 8A shows an alternative example of a tubular member including a distensible bumper in a relaxed or expanded configuration.

FIGS. 8B and 8C show the tubular member of FIG. 8A with an instrument advanced into a lumen of the tubular member to distend and reduce an outer cross section of the bumper to facilitate removal.

FIG. 9A is a perspective view of another example of a low profile gastrostomy tube device that may be implanted within a patient's abdomen.

FIGS. 9B and 9C are side and cross-sectional views, respectively, of the device of FIG. 9A.

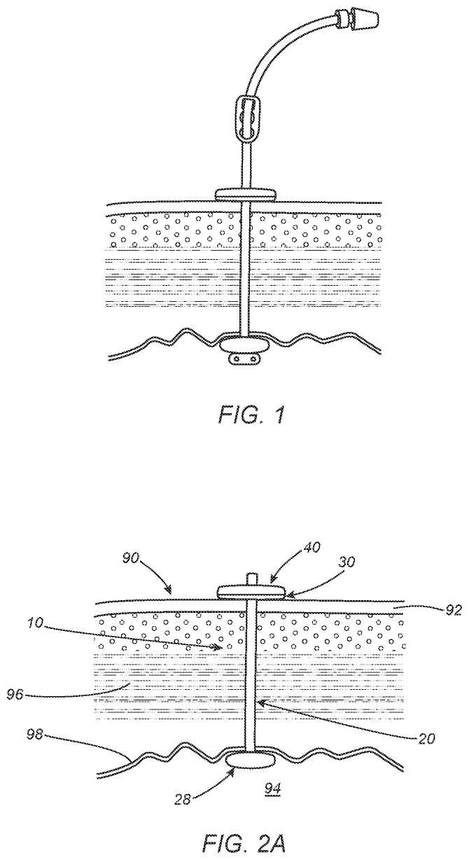

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise.

Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

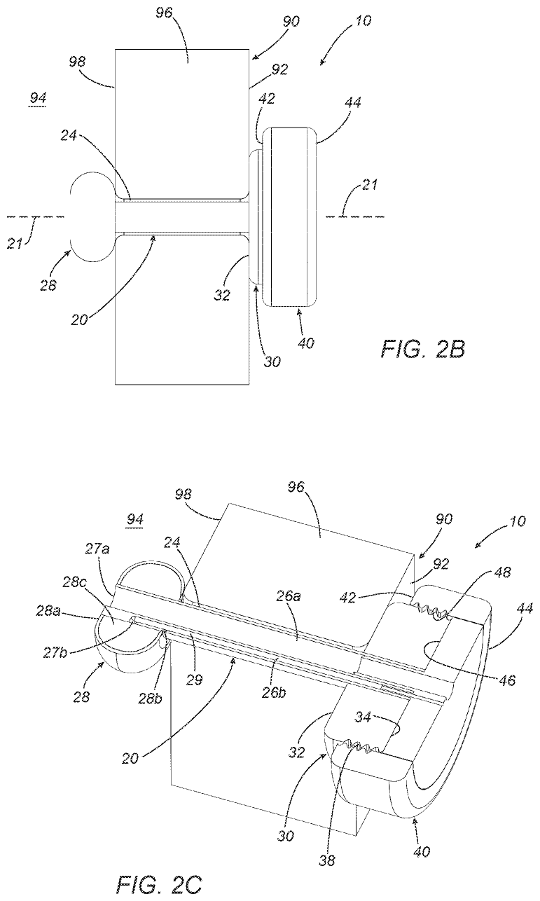

Turning to the drawings, FIGS. 2A-2C show an example of a gastrostomy tube device 10 implanted within an abdomen of a patient 90. As shown, the device 10 generally includes an elongate tubular member or body 20, an expandable member or other bumper 28, a bolster, 30, and an adapter 40. The components of the device 10 may be provided to a medical practitioner separately, e.g., as a kit, that may be assembled during an implantation procedure, as described elsewhere herein, to provide a low-profile gastrostomy tube implanted within the abdomen of a patient 90, i.e., between the skin 92 and stomach 94, e.g., for a patient who cannot take in nutrients by mouth for an extended period of time.

In addition or alternatively, the device 10 may be included in a system, e.g., including one or more additional tools or instruments to facilitate implantation and/or removal of the device 10, as described further elsewhere herein. For example, the system may include one or more guidewires, guide sheaths, pusher members, and/or cutters (not shown) for introducing the tubular member 20 into a patient's body 90 and/or cutting the tubular member 20 during implantation. In addition, the system may include one or more sources of inflation media and/or vacuum for expanding and/or collapsing the bumper 28, e.g., if the bumper 28 is a balloon, and/or one or more tools for otherwise collapsing the bumper 28 (also not shown) for subsequent removal, as described further elsewhere herein.

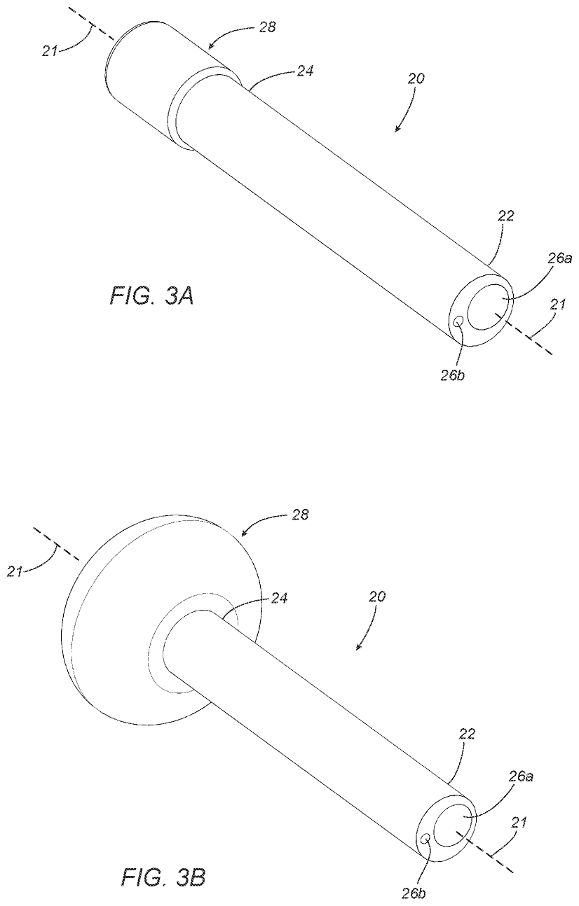

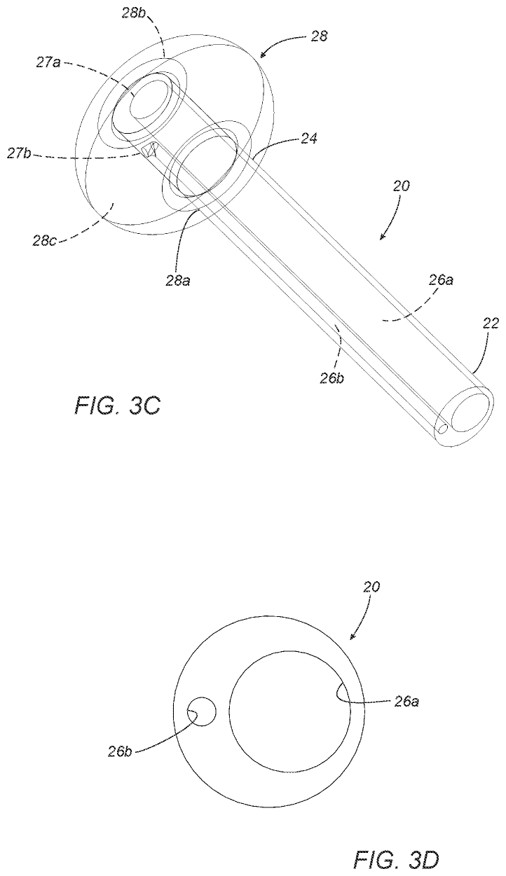

With additional reference to FIGS. 3A-3D, the tubular member 20 includes a first end 22, e.g., having a raw or tapered edge or other low profile cross-section no larger than the main length of the tubular member 20, e.g., to facilitate introduction through abdominal tissue 96 between the stomach 94 and skin 92, a second end 24 including the bumper 28 thereon, and defining a longitudinal axis 21 therebetween. The tubular member 20, as provided in a kit, may have an original length between the first and second ends 22, 24 sufficient such that the first end extends from the patient's skin 92 when the second end 24 is positioned within the patient's stomach 94 with the bumper 28 against the wall 98 of the stomach 94 and the tubular member 20 extends through the intervening tissue 96 to the skin 92. In one example, the tubular member 20 may have a substantially uniform outer diameter between the first and second ends 22, 24, e.g., between about four and eight millimeters (4.0-8.0 mm), and an original length between the ends 22, 24 between about twenty and one hundred centimeters (20-100 cm) or about seventy five centimeters (75 cm) or more, which may facilitate introduction and/or manipulation of the tubular member 20 until the first end 22 is exposed from the patient's abdomen. After excess material is cut or otherwise removed, the final length of the tubular member 20 between the second end 24 and the severed first end may be between about one and five centimeters (1.0-5.0 cm). Optionally, an original length, e.g., between about twenty and one hundred centimeters (20-100 cm), may also be retained for a period of time, e.g., from several hours to several months or about one or two days while the device is used and/or before excess material is cut or otherwise removed. For example, the final length may be such that the first end 22 extends between about half and five centimeters (0.5 to 5 cm) from the skin 92 after the tubular member 20 is introduced into the stomach 94, the bumper 28 is positioned against the wall 98, and the excess material is removed, as described further elsewhere herein. The tubular member 20 may be formed from flexible, biocompatible material, e.g., plastic, such as polyurethane, silicone, and the like, to facilitate introduction into the stomach 94 and having sufficient rigidity to prevent collapse during use.

In addition, the tubular member 20 includes one or more lumens, e.g., a primary or feeding lumen 26a and, optionally one or more secondary lumens, e.g., inflation lumen 26b, extending between the first and second ends 22, 24, as best seen in FIGS. 3C and 3D. In the example shown in FIGS. 3A-3D, the bumper 28 is an annular balloon permanently attached around or otherwise to the second end 24 of the tubular member 20, e.g., such that the feeding lumen 26a extends between an opening 27a in the second end 24 and the first end 22 of the tubular member 20, i.e., through the balloon 28. For example, as best seen in FIG. 2C, the balloon 28 may include a first end 28a attached to the tubular member 20, e.g., immediately adjacent the opening 25b, and a second end 28b attached to the outer wall of the tubular member 20 a distance away from the first end 28a to define an annular interior 28c, e.g., by one or more of bonding with adhesive, sonic welding, fusing, interference fit, and the like.

In this example, the tubular member 20 includes inflation lumen 26b extending at least partially between the first and second ends 22, 24, e.g., extending from the first end 22 adjacent the feeding lumen 26a and communicating with the interior 28c of the balloon 28 via one or more ports., e.g., port 27. The balloon 28 may be formed from inelastic material, such as PET, nylon, polyurethane, silicone, and the like, such that the balloon 28 expands to a predetermined size and/or shape when inflated, e.g., a relatively flat donut shape to facilitate placement against the wall 98 of the stomach 94 and prevent removal through the tissue 96. Alternatively, the balloon 28 may be formed from elastic material, e.g., silicone or other elastomeric material, such that the size and/or shape of the balloon 28 may be dependent upon the volume and/or pressure of fluid introduced into the interior 28c.

Optionally, a valve 29 may be provided within the inflation lumen 26b, e.g., adjacent the second end 24, spaced apart from the first end 22 to remain when a portion of the first end 22 is removed, or otherwise communicating with the inflation lumen 26b such that inflation media, e.g., gas such as carbon dioxide or liquid such as saline, may be delivered through the valve 29, e.g., from a syringe, pump, and the like (not shown) and inflation lumen 26a into the interior 28c to inflate the balloon 28. The valve 29 may prevent the inflation media from subsequently escaping thereby keeping the balloon 28 inflated indefinitely (e.g., until the device 10 is removed from the patient, as described elsewhere herein). For example, the valve 29 may be a one-way valve, such as a flap or duck-bill valve, that may open when inflation media is introduced into the inflation lumen 26b, i.e., under positive pressure from the first end 22, and automatically close when pressure from the inflation media is discontinued, thereby containing the inflation media within the interior 28c and preventing subsequent leakage.

Optionally, the valve 29 may be opened mechanically, e.g. by introducing a needle or other tool into the inflation lumen 26b from the first end 22, thereby allowing the inflation media within the interior 28c to escape, e.g., to deflate the balloon 28 for removal. Alternatively, the valve 29 may be configured to fail or otherwise open when a vacuum is applied to the inflation lumen 26b, i.e., under negative pressure from the first end 22, that is above a predetermined threshold. For example, a syringe or vacuum line (not shown) may be connected to the inflation lumen 26b to apply sufficient vacuum to open the valve 29 and evacuate inflation media from the balloon 28. In a further alternative, the balloon 28 may include one or more regions configured to fail or otherwise open when additional positive pressure is applied to the interior 28c above a predetermined threshold. For example, a weakened region (not shown) may be provided in a wall of the balloon 28 that may fail after the balloon 28 is initially inflated, i.e., when sufficient additional positive pressure is added, e.g., from a syringe or other source of additional inflation media to facilitate removal of the device 10.

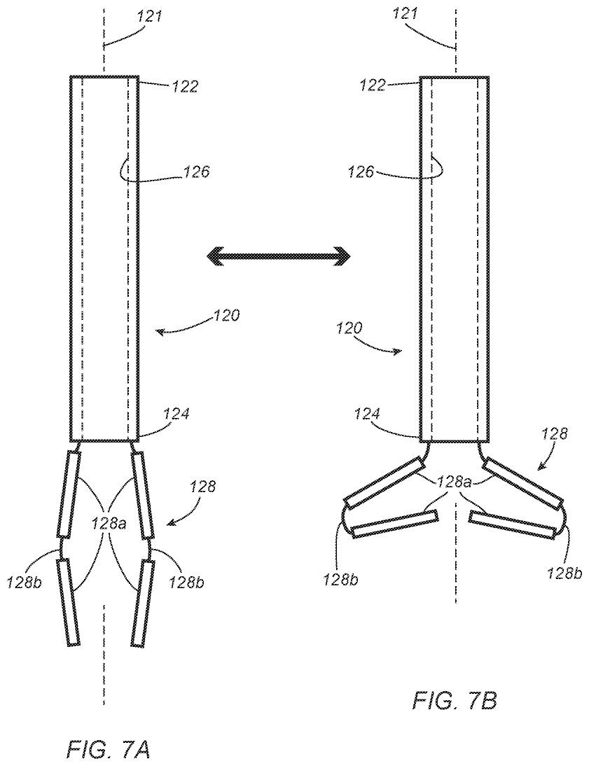

Alternatively, as shown in FIGS. 7A and 7B, a tubular member 120 may be provided that includes first and second ends 122, 124 and a feeding lumen 126, e.g., generally similar to other examples herein, but including a bistable or other mechanically expandable device for a bumper 128. The bumper 128 may be directed between a collapsed configuration, e.g., having a profile no larger than the second end 124, and an expanded configuration, e.g., wherein one or more elements extend radially outwardly from the second end 24. For example, the bumper 128 may include a plurality of mechanically expandable elements 128a mounted or otherwise attached to the second end 124 that may be directed between a collapsed configuration, e.g., in which the elements 128a extend substantially axially relative to longitudinal axis 121, e.g., as shown in FIG. 7A, and an expanded configuration, e.g., in which at least a portion of the elements extend transversely, e.g., substantially perpendicular to the axis 121 as shown in FIG. 7B.

In the example, shown, each of the elements 128a may include an intermediate region 128b that may buckle or fold outwardly relative to the axis 121 to define the expanded configuration. Alternatively, one or more flanges, wings, or other features that may be provided that may simply fold, bend, or otherwise deflect outwardly from the second end 124 towards the expanded configuration. The elements 128a may be biased towards the expanded configuration such that the elements 128a may be constrained in the collapsed configuration, e.g., by one or more of an external sleeve, one or more filaments, and/or other constraints (not shown) during introduction. In this example, the elements 128a may automatically expand when released from the constraint(s), e.g., within the stomach such that the elements 128a may be pulled against the wall of the stomach to stabilize the second end 124. Alternatively, the elements 128a may be expanded by an actuator or a separate tool (not shown) that may plastically deform the elements 128a outwardly to the expanded configuration.

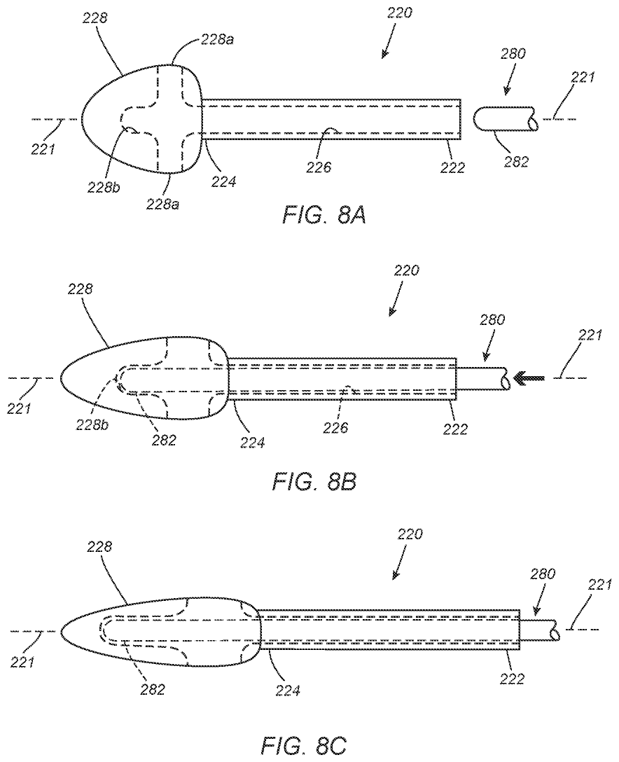

In a further alternative, the bumper may have a fixed shape, e.g., defining an outer diameter or cross-section that is larger than the tubular member yet small enough to be directed through a subject upper gastrointestinal tract into the stomach. For example, as shown in FIG. 8A, a tubular member 220 may be provided that includes first and second ends 222, 224 and a feeding lumen 226 extending therebetween, generally similar to other examples herein, and a distensible bumper 228 may be provided on the second end 224. The bumper 228 may be formed from flexible material, e.g., silicone or other elastomeric material, such that the bumper 228 is biased to an expanded or relaxed shape, such as that shown in FIG. 8A. The material may allow the bumper 228 to be positioned against the wall of the stomach, e.g., providing a shoulder to abut the wall 98 of the patient's stomach 94 to stabilize the tubular member 220 and prevent removal through the abdominal tissue 96 after implantation.

However, if desired, the bumper 228 may be distended to reduce its outer cross-section. For example, as shown in FIGS. 8B and 8C, a tool 280 may be provided including a rounded or other atraumatic distal end 282 sized for introduction into the lumen 226. During removal of the tubular member 220 (e.g., after removing the bolster and adapter not shown, on the first end 222), the distal end 282 of the tool 280 may be inserted into the lumen 226 until it contacts the bumper 228 and then advanced further to cause the bumper 228 to stretch along the axis 221. Given the elasticity of the material, as the bumper 228 distends, its outer profile is reduced, e.g., towards the diameter of the second end 224 of the tubular member 220, which may facilitate removal through abdominal tissue. In the example shown, the bumper 228 includes one or more internal passages, e.g., outlets 228a for delivering nutrients into the stomach through the lumen 226 and a recess 228b to receive the distal end 282 of the tool 280, e.g., to ensure that the bumper 228 distends substantially uniformly.

Optionally, the tool 280 may include one or more features to facilitate distending the bumper 228 without applying excessive force that may cause the bumper 228 to tear or otherwise fail. For example, the tool 280 may include an indicator on its proximal end (not shown), such as a light, a speaker, and the like, that may be coupled to a sensor measuring the distal force applied to the distal end 282. If the force applied to the bumper approaches or exceeds a maximum force, the indicator may provide an output to the user to notify the user not to apply additional force to prevent failure of the bumper 228. In addition or alternatively, a spring or other limiter may be provided on the proximal end such that, when a maximum force is exceeded, the spring or limiter may deform or be released to prevent the excess force from being translated to the distal end 282.

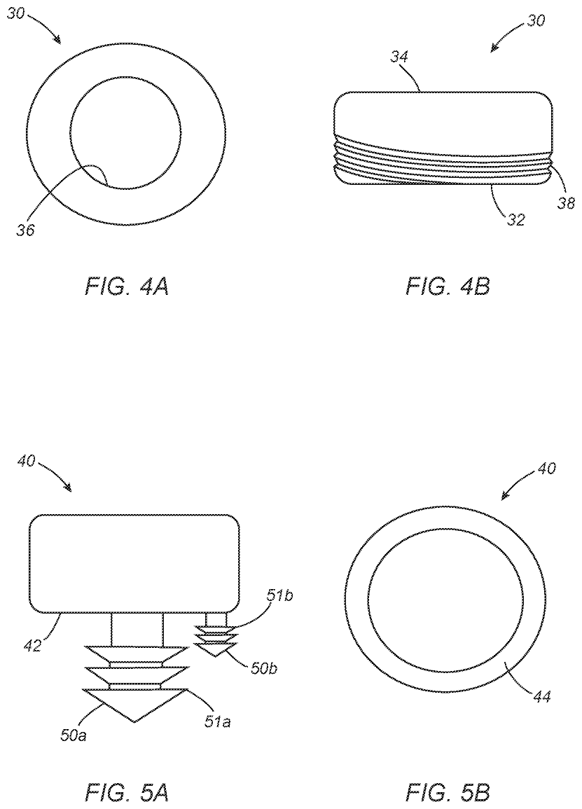

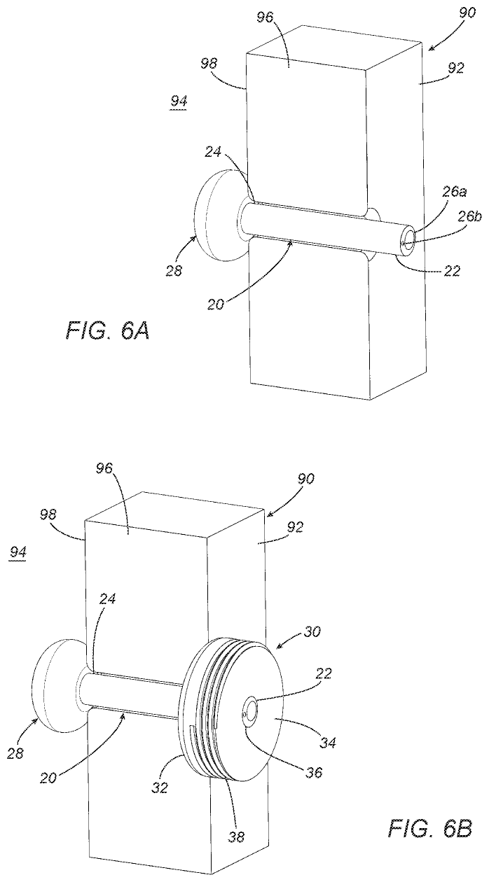

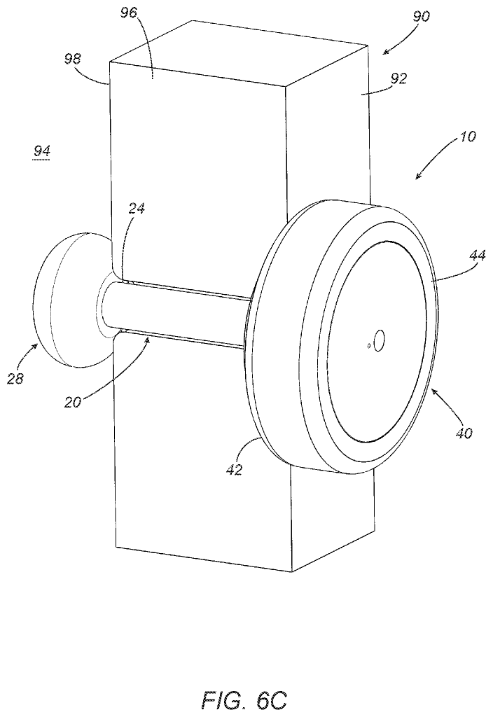

Turning to FIGS. 4A and 4B, the bolster 30 includes a ring or other annular housing that is connectable around or otherwise to the tubular member 20, e.g., after the first end 22 has been directed from within the patient's stomach 94 through intervening tissue 96 and extends from the patient's skin 92, e.g., as shown in FIGS. 6A and 6B. Generally, as shown, the bolster 30 includes a first or lower end 32 for placement against the skin 92, a second or upper end 34, and a passage 36 therebetween that is sized to be received over the first end 22 of the tubular member 20. In one example, the bolster 30 may be attached to the tubular member 20 by one or more of crimping the bolster 30 down onto the tubular member 20, bonding with adhesive, one or more ratchet or detent elements that may allow the bolster 30 to be advanced over the first end 22 but not removed, and the like. In addition or alternatively, a zip-tie or other clamp (not shown) may be applied around a portion of the bolster 30 that may apply a radially inward force to secure the bolster 30 to the tubular member 20, e.g., as described elsewhere herein with respect to FIGS. 8A-8C.

Turning to FIGS. 5A and 5B, the adapter 40, in turn, is connectable to the bolster 30 and/or tubular member 20, e.g., after excess material of the tubular member 20 beyond the bolster 30 has been separated, as described further elsewhere herein. In the example shown, the adapter 40 includes a first or lower end 42 and a second or upper end 44 and one or more features for coupling to the tubular member 20. The bolster 30 and adapter 40 may include cooperating connectors for permanently or removably securing the adapter 40 to the bolster 30. For example, as shown in FIGS. 2C, 4B, and 6B, the bolster 30 may include one or more external threads 38 and the adapter 40 may include a recess 46 including internal threads 48, thereby allowing the adapter 40 to be threaded around and/or over the bolster 30, as shown. Alternatively, other mechanisms may be used, such as one or more of snap connectors, adhesive, screws, pins, cooperating magnets, and the like to secure the adapter 40 to the bolster 30 and/or the tubular member 20, such as the connectors provided on the device 310 described further below.

Optionally, as shown in FIG. 5A, the adapter 40 may include one or more features that engage with the tubular member 20, e.g., after cutting or otherwise removing excess material from the first end 22. For example, as shown, the adapter 40 may include a first tubular hub 50a extending from the lower surface 42 sized to be inserted into the feeding lumen 26a at the severed end of the tubular member 20. Optionally, the hub 50a may include one or more connectors, e.g., annular ratchet elements 51a, and the like, that may allow the hub 50a to be inserted into the first end 22 while preventing removal. Optionally, as also shown in FIG. 5A, a second hub 50b may be provided that is similarly insertable into the inflation lumen 26b, e.g., also including one or more ratchet elements 51b or other connector.

The adapter 40 may include a connector for removably connecting to a feeding tube (not shown). For example, the adapter 40 may include a port or fitting for slidably and/or rotatably receiving an end of a feeding tube, e.g., including a valve (not shown) that is biased to a closed position to prevent leakage from the feeding lumen 26a, but may be opened when a feeding tube is coupled to the port. Alternatively, cooperating connectors may be provided that may be threaded or otherwise coupled together to provide a fluid-tight connection such that nutrients may be delivered through the feeding tube and the feeding lumen 26a into the patient's stomach 94. Such connectors may include a release mechanism, e.g., such that, if a predetermined force is applied to the feeding tube, the connectors will automatically release or fail to prevent the force from being transmitted to the gastrostomy tube device 10.

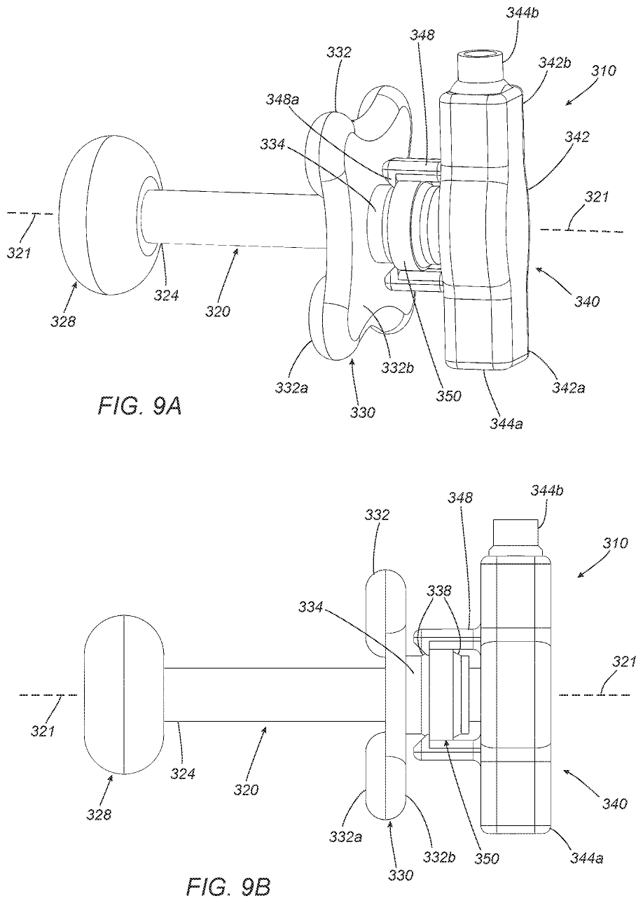

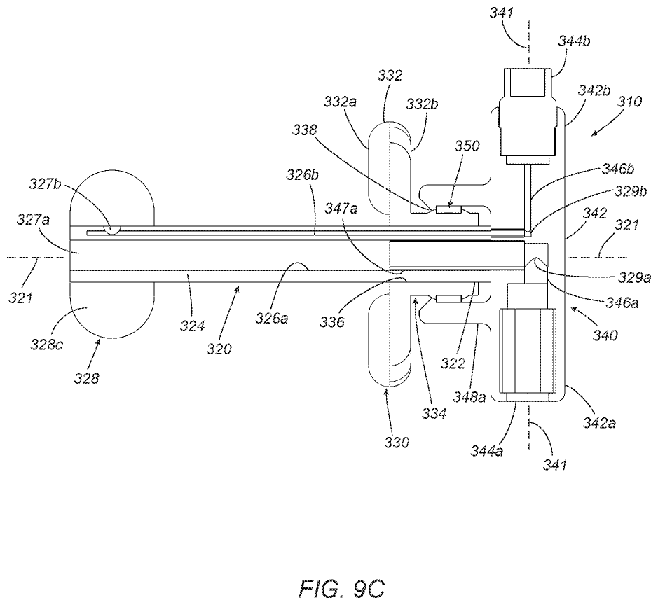

Turning to FIGS. 9A-9C, another example of a device 310 is shown that includes a tubular member 320, a bumper 328, a bolster 330, and an adapter 340, generally similar to the device 10. For example, similar to other examples herein, the tubular member 320 may include a first end 322, a second end 324 including the bumper 328, and one or more lumens extending therebetween. For example, the tubular member 320 may include a primary or feeding lumen 326a extending from the first end 322 to an outlet 327a in the second end 324 beyond the bumper 328. As shown, the bumper 328 is a balloon attached to the second end 324, and the tubular member 320 also includes a secondary or inflation lumen 326b extending from the first end 322 to a port 327b communicating with an interior 328c of the balloon 328.

The bolster 330 includes a base plate 332 shaped for placement against the patient's skin, e.g., including a lower surface 332a including one or more spacers or other features configured to contact the skin, and a substantially flat or lower profile upper surface 332b. A nipple or port 334 extends from the base plate 332 including an internal passage 336 therethrough sized to be received over the first end 322 of the tubular member 320. The nipple 334 may include a continuous annular wall that may be sufficiently flexible such that nipple 334 may be compressed inwardly around the first end 322. Alternatively, the nipple 334 may include a plurality of tabs (not shown) extending from the base plate 332 that are spaced apart circumferentially from one another to surround the passage 336. In this alternative, the tabs may be sufficiently flexible to be bent or otherwise compressed inwardly to secure the bolster 330 around the tubular member 320.

For example, as shown, a clamp or other securing device 350 may be provided that is sized to be received around the nipple 334 and then used to compress the nipple 334 inwardly to secure the bolster 330 to the first end 322 of the tubular member 320. For example, the clamp 350 may include a “C” shaped or flexible elongate member including two ends that include a ratchet or other mechanism (not shown) allowing the ends to be engaged together after placing the clamp 350 around the nipple 334, and tightened to compress the nipple 334 inwardly. For example, a zip-tie device may be provided that includes a receptacle on a first end that may receive a second, opposite end after wrapping the zip-tie around the nipple 334. The ends may include cooperating ratchets, detents, or other features that allow the ends to be tightened and prevent release to clamp the nipple 334 around the first end 320. Optionally, the second end may include one or more weakened regions to allow excess material to be separated by pulling or by cutting to provide a low profile clamp 350 around the nipple 334.

The adapter 340 includes a housing 342 that may be secured to the bolster 330 to provide connectors for coupling a feeding tube and/or source of inflation media to the device 310. For example, as shown, the housing 342 may have an elongate shape including first and second ends 342a, 342b defining a housing axis 341 therebetween. Each end 342a, 342b includes a connector 344a, 344b for coupling to a feeding tube and a source of inflation media (not shown), respectively. Each connector 344a, 344b communicates with a respective passage 346a, 346b within the housing 342 configured to communicate, in turn, with respective lumens 326a, 326b of the tubular member 320.

Optionally, as shown in FIG. 9C, a tubular extension 347a may extend from the housing 342, e.g., substantially perpendicular to the axis 341 that communicates with the passage 346a and is sized to be inserted into the feeding lumen 326a. For example, a section of hypotube or other rigid tubular material may be attached to or otherwise extend from the housing 342 that be inserted into the feeding lumen 326a to support the lumen 326a, e.g., when the clamp 350 and nipple 334 are compressed around the first end 322 of the tubular member 320, to prevent collapse of the feeding lumen 326a. In addition or alternatively, if desired, a tubular extension (not shown) may also be provided that communicates with passage 346b and is sized to be inserted into the inflation lumen 326b of the tubular member 320.

Optionally, one or both passages 346a, 346b may include a valve 329a, 329b. For example, a one-way valve 329a may be provided in the passage 346a from the feeding tube connector 342a that may be biased closed to prevent fluid from escaping from the patient's stomach through the feeding lumen 326a after implantation of the device 310. When a feeding tube (not shown) is connected to the connector 344a to introduce nutrients into the patient's stomach, the valve 329a may open under positive pressure, e.g., during delivery of the nutrients, and then close automatically when delivery is discontinued. Alternatively, a valve may be provided in the connector 342a that may be opened when a connector from the feeding tube is connected to the adapter 340 and automatically close when the feeding tube is removed. Similarly, a valve 329b may be provided for the inflation lumen 326b, e.g., rather than providing a valve within the tubular member 320, as described with other devices herein.

In addition, the housing 342 includes one or more connectors 348 for securing the adapter 340 to the bolster 330, e.g., a pair of arms 348 extending from the housing 342 substantially perpendicular to the housing axis 341. The arms 348 may include one or more detents or other features 348a that may be configured to engage with corresponding features 338 on the nipple 334 of the bolster 330. For example, the detents 348a may snap over annular ridges 338 on the nipple 334 and/or over the edge of the clamp 350 to secure the adapter 350 to the bolster 340. With the arms 348 engaged to the nipple 334 and/or clamp 350, the adapter 350 may be secured such that the housing axis 341 is substantially perpendicular to the longitudinal axis 321 of the tubular member 320. Thus, the adapter 350 may be secured relatively close the patient's skin in a low profile orientation that may increase comfort and/or convenience for the patient and/or minimize risk of the adapter 350 being accidentally separated from the bolster 330.

Turning to FIGS. 6A-6C, the devices herein may be implanted in a patient's abdomen to provide a low profile gastrostomy tube that may be used to deliver nutrients directly into the patient's stomach. For example, the device 10 shown in FIGS. 2A-2C may be implanted into an abdomen of a patient 90, i.e., extending from the patient's skin 92 through abdominal tissue 96 into the stomach 94, with the bolster 30 and adapter 40 providing a low profile against the patient's skin 92 while the bumper 28 is positioned within the stomach 94.

In an exemplary method for implantation, a first end of a guidewire (not shown) may be directed through the abdomen of the patient 90 from skin 92 overlying the patient's stomach 94 through intervening tissue 96 into the stomach 94, e.g., using conventional procedures. For example, a needle (also not shown) may be directed directly through the skin 92 and intervening tissue 96 and into the stomach 94, and the guidewire may be introduced through a lumen of the needle. The first end of the guidewire may then be directed along the patient's upper gastrointestinal tract from the patient's stomach 94 and out the patient's mouth (not shown).

As shown in FIGS. 3A and 3B, the bumper 28 may be expanded or otherwise provided in an expanded configuration larger than the outer diameter of the tubular member 20, and then the first end 22 of the tubular member 20 may be introduced using the guidewire into the patient's mouth and upper gastrointestinal tract until the tubular member 20 is positioned within the patient's stomach 94. For example, the first end 22 of the tubular member 20 may be connected to the first end of the guidewire outside the patient's mouth, and the guidewire may be pulled, e.g., from a second opposite outside the patient's abdomen to direct the tubular member 20 into the stomach 94. Alternatively, the tubular member 20 may be advanced over the guidewire, e.g., using a tubular pusher, guide, or other instrument (not shown).

With additional reference to FIG. 6A, the first end 22 of the tubular member 20 may then be directed through the intervening tissue 96 until the first end 22 is exposed outside the patient's abdomen, e.g., with the bumper 28 positioned against the anterior wall 98 of the stomach 94. Optionally, one or more dilators and/or other tubular devices may be inserted through the tissue 96, e.g., over the guidewire, if desired to dilate the tissue 96 and/or otherwise accommodate inserting the first end 22 through the tissue 96 until disposed outside the abdomen. Alternatively, if the bumper 28 is a balloon or otherwise expandable, the tubular member 20 may be introduced and positioned through the intervening tissue 96 with the bumper collapsed. With the first end 22 exposed, a source of inflation media (not shown) may be coupled to the inflation lumen 26b and inflation media introduced into the interior 28c of the balloon 28 to inflate the balloon 28 before the bumper 28 is positioned against the wall 98 of the stomach 94.

The bolster 30 may then be secured to the tubular member 20 against the patient's

skin 92, e.g., advanced over the first end 22 such that the intervening tissue 96 is sandwiched between the bumper 28 and the bolster 30. Excess length of the tubular member 20 exposed beyond the bolster 30 may be cut or otherwise separated, e.g., such that the tubular member 20 is flush with the bolster 30, e.g., as shown in FIG. 6B, and then the adapter 40 may be secured to the bolster 30, e.g., as shown in FIG. 6C. Similarly, the bolster 330 shown in FIGS. 9A-9C may be advanced over the exposed first end 322 of the tubular member 320, excess material removed, and then the adapter 340 may be secured to the bolster 330. Optionally, after initially positioning the bolster 330 over the first end 322 of the tubular member 320, the clamp 350 may be positioned around the nipple 334 and partially tightened. Thus, the clamp 350 may be used to initially stabilize the tubular member 320 while the excess material is removed. After coupling the adapter 340 to the bolster, the clamp 350 may then be tightened further to compress the nipple 334 around the tubular member 320. If a tubular extension 347a is provided on the adapter 340, the extension 347a may be inserted into the feeding lumen 326a before the clamp 350 is tightened further, thereby preventing collapse of the feeding lumen 326a.

When desired, a feeding tube may be connected to the adapter 40, 342 to provide nutrients to the patient. When the patient no longer needs a gastrostomy tube, the device 10, 310 may be removed, e.g., by collapsing the bumper 28, 328 and removing the tubular member 20, 320 from the patient's abdomen. For example, where the bumper 28 is a balloon, as shown in FIGS. 6A-6C, an instrument (not shown) may be introduced through the patient's upper gastrointestinal tract into the stomach 94 and used to puncture or otherwise break the balloon 28. The tubular member 20 may then be pulled from the bolster 30 and/or adapter 40 (still secured to the first end 22 of the tubular member 20) to direct the second end 24 from the stomach 94 through the intervening tissue 96 or, if desired, one or both of the bolster 30 and/or adapter 40 may be removed from the tubular member 20 before pulling the tubular member 20.

Alternatively, if the balloon 28 includes a weakened region, additional inflation media may be introduced into the interior 28c of the balloon 28 until the weakened region fails, thereby allowing the inflation media to escape and the balloon 28 to collapse.

In another alternative example, where the bumper 28 is a balloon communicating with an inflation lumen 26b of the tubular member 20, an instrument may be directed through valve 29 into the inflation lumen 26b to remove inflation media from the balloon using the instrument or the valve 29 may be opened to allow the inflation media to escape and collapse the balloon. Alternatively, a vacuum may be delivered into the inflation lumen 26b, e.g., by applying suction from a syringe or vacuum line (not shown) that is above a predetermined threshold, to cause the valve 29 to fail and allow inflation media to escape and collapse the balloon 28. The remaining tissue tract may then be closed using conventional methods.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A gastrostomy tube device for implantation within an abdomen of a patient, comprising:
an elongate tubular member comprising a first end sized for introduction into the patient's mouth, a second end including an expandable member, and a feeding lumen extending between the first and second ends;
a bolster connectable around the first end after the first end has been directed from within the patient's stomach through intervening tissue and extends from the patient's skin; and
an adapter connectable to the bolster after the first end and excess material of the tubular member beyond the bolster has been separated, the adapter comprising a connector for removably connecting to a feeding tube.

2. The device of claim 1, wherein the tubular member has a length between the first and second ends such that the first end extends from the patient's skin when the first end is directed from within the patient's stomach through intervening tissue and the expandable member is positioned against an anterior wall of the stomach.

3. The device of claim 1, wherein the expandable member comprises an annular balloon on the second end.

4. The device of claim 3, wherein the tubular member further comprises an inflation lumen extending from the first end and communicating with an interior of the balloon.

5. The device of claim 4, further comprising a valve communicating with the inflation lumen to prevent inflation media from escaping from the interior of the balloon after the balloon is inflated.

6. The device of claim 5, wherein the valve allows inflation when positive pressure is applied and deflation when vacuum pressure is applied above a predetermined threshold.

7. The device of claim 4, further comprising a weakened region in one of the balloon and the inflation lumen configured to fail or open permanently when positive or vacuum pressure above a predetermined threshold is applied to allow the balloon to be collapsed for removing the device from the subject's abdomen.

8. The device of claim 1, wherein the expandable member comprises a bistable element that is expandable radially outwardly from the second end of the tubular member.

9. The device of claim 1, wherein the bolster and the adapter comprise cooperating connectors for securing the adapter to the bolster.

10. (canceled)

11. The device of claim 9, wherein the cooperating connectors comprise one or more of snap connectors, adhesive, screws, pins, cooperating magnets, and mating threads that allow the adapter to be threaded around the bolster.

12. The device of claim 9, wherein the bolster comprises a base plate configured for placement against the patient's skin and a nipple extending from the base plate, and wherein the cooperating connectors comprise a plurality of arms extending from a housing of the adapter and one or more elements on the nipple.

13. The device of claim 1, wherein the bolster comprises a base plate configured for placement against the patient's skin and a nipple extending from the base plate, the device further comprising a clamp configured for positioning around the nipple to compress the nipple inwardly around the tubular member.

14. (canceled)

15. A kit for implanting a gastrostomy tube within an abdomen of a patient, comprising:
an elongate tubular member comprising a first end sized for introduction into the patient's mouth, a second end including an expandable member, and a feeding lumen extending between the first and second ends;
a bolster connectable around the first end after the first end has been directed from within the patient's stomach through intervening tissue and extends from the patient's skin; and
an adapter connectable to the bolster after the first end and excess material of the tubular member beyond the bolster has been separated, the adapter comprising a connector for removably connecting to a feeding tube.

16. The kit of claim 15, wherein the tubular member has a length between the first and second ends such that the first end extends from the patient's skin when the first end is directed from within the patient's stomach through intervening tissue and the expandable member is positioned against an anterior wall of the stomach.

17. The kit of claim 15, wherein the expandable member comprises an annular balloon on the second end.

18. The kit of claim 17, wherein the tubular member further comprises an inflation lumen extending from the first end and communicating with an interior of the balloon.

19. The kit of claim 18, further comprising a valve communicating with the inflation lumen to prevent inflation media from escaping from the interior of the balloon after the balloon is inflated.

20. The kit of claim 19, wherein the valve allows inflation when positive pressure is applied and deflation when vacuum pressure is applied above a predetermined threshold.

21-30. (canceled)

31. A system for implanting a gastrostomy tube within an abdomen of a patient, comprising:
an elongate tubular member comprising a first end sized for introduction into the patient's mouth, a second end including an expandable member, a feeding lumen extending between the first and second ends, and an inflation lumen extending from the first end and communicating with an interior of the expandable member;
a bolster connectable around the first end after the first end has been directed from within the patient's stomach through intervening tissue and extends from the patient's skin;
an adapter connectable to the bolster after the first end and excess material of the tubular member beyond the bolster has been separated, the adapter comprising a connector for removably connecting to a feeding tube; and
a source of inflation media connectable to the inflation lumen for introducing inflation media into the interior to expand the expandable member.

32-50. (canceled)

51. A method for implanting a gastrostomy tube into an abdomen of a patient, comprising:
providing an elongate tubular member comprising a first end, a second end including an expandable member, and a lumen extending between the first and second ends;
directing a first end of a guidewire through the abdomen of the patient from skin overlying the patient's stomach through intervening tissue into the stomach;
directing the first end of the guidewire along the patient's upper gastrointestinal tract from the patient's stomach and out the patient's mouth;
expanding the expandable member on the second end of the tubular member;
introducing the first end of the tubular member using the guidewire into the patient's mouth and upper gastrointestinal tract until the tubular member is positioned within the patient's stomach;
directing the first end of the tubular member through the intervening tissue until the first end is exposed outside the patient's abdomen;
securing a bolster to the first end adjacent the patient's skin such that the intervening tissue is sandwiched between the expandable member within the stomach and the bolster;
separating excess length of the tubular member exposed beyond the bolster; and
securing an adapter to the bolster, the adapter comprising a connector for removably connecting a feeding tube to the adapter.

52-76. (canceled)
